(19) United States
(12) Patent Application Publication
(10) Pub. No.: US 2007/0299346 A1
(43) Pub. Date: Dec. 27, 2007

(54) Method, system, and apparatus for a doppler catheter

(76) Inventor: Paul Bigeleisen, Pittsford, NY (US)

Correspondence Address: BLANK ROME LLP, 600 NEW HAMPSHIRE AVENUE, N.W., WASHINGTON, DC, 20037, US

(21) Appl. No.: 11/812,778

(22) Filed: Jun. 21, 2007

Related U.S. Application Data
(60) Provisional application No. 60/815,573, filed on Jun. 22, 2006.

(51) Int. Cl. A61B 8/06

(52) U.S. Cl. 600/468

(57) ABSTRACT

A Doppler catheter assembly includes a catheter having an inner lumen and an outer lumen, a first piezoelectric element, and a second piezoelectric element. The first piezoelectric element is adapted to be positioned proximate to the second piezoelectric element about the inner lumen, thereby forming a transmitter/receiver.

### FIELD OF THE INVENTION

The present invention is directed to a catheter for use in monitoring blood flow. In more detail, the present invention uses Doppler technology to detect the occurrence of venous air emboli (VAE) during surgery.

### DESCRIPTION OF THE RELATED ART

VAE are a potentially lethal complication of some surgical procedures. VAE may occur when gas enters the systemic venous system at the surgical site. VAE generally occur when a body part is elevated above the heart and the venous lumen is open to the atmosphere approximately simultaneously. In more detail, VAE may occur when a pressure gradient develops that favors entry of air into the venous system.

Examples of air entry sites include veins in the bones of the skull or vertebral column, veins in the bones of extremities, cerebral venous sinuses, vertebral epidural veins, and veins in pelvic ligaments. The accidental entry of air may also be caused by an air powered drill, air insufflation during laparoscopy, or when using an air filled syringe for identification of epidural space, as non-limiting examples.

Current VAE treatments include precordial Doppler and central venous catheter aspiration. However, for a variety of reasons, these methods of detection are unsatisfactory.

Generally, precordial Doppler is placed in the right or left parasternal location in the second to fourth intercostal spaces. The presence of air may be signaled by the change in sound on the monitor from a regular high pitched swishing to an erratic roar. However, by the time the embolism is detected using precordial Doppler, it is often impossible to retrieve it. As a result, the VAE may travel to the brain, heart, or pulmonary artery and cause death.

Another problem with precordial Doppler is its relatively high rate of error, with respect to both false positives and false negatives.

### SUMMARY OF THE INVENTION

The present invention was made in view of the difficulties associated with the techniques of the related art.

### BRIEF DESCRIPTION OF THE DRAWINGS

Exemplary embodiments of the present invention are described below with reference to the drawings, in which:

FIG. 1 is an illustration of an exemplary situation in which the present invention is configured to be used;

FIG. 2 illustrates an exemplary configuration of the Doppler catheter according to an aspect of the present invention;

FIG. 3 illustrates an exemplary configuration of the Doppler catheter according to another aspect of the present invention;

FIGS. 4(a) and 4(b) illustrate exemplary configurations of the piezoelectric elements;

FIG. 5 illustrates an exemplary system into which the present invention may be incorporated;

FIGS. 6(a) and 6(b) illustrate exemplary configurations of the Doppler catheter according to non-limiting embodiments of the present invention;

FIG. 7 illustrates the results achieved during experimentation with different configurations of the Doppler catheter of the present invention, in which “number” represents the number of bubbles and “distance in cm” represents a distance between the Doppler assembly and the bubble source;

FIG. 8 illustrates exemplary results for percentages of bubbles detected as a function of size, where “number” represents a number of bubbles;

FIG. 9 illustrates acoustic pressure along the axial and radial axes as a function of distance, the distance being the distance between the Doppler assembly and a hydrophone;

FIG. 10 illustrates experimental results reflecting the influence of spacing between transducer elements of the sensitivity of the Doppler probe;

FIG. 11 illustrates an exemplary configuration for a test system according to the present invention; and

FIG. 12 illustrates an additional exemplary test system according to the present invention.

### DESCRIPTION OF THE PREFERRED EMBODIMENTS

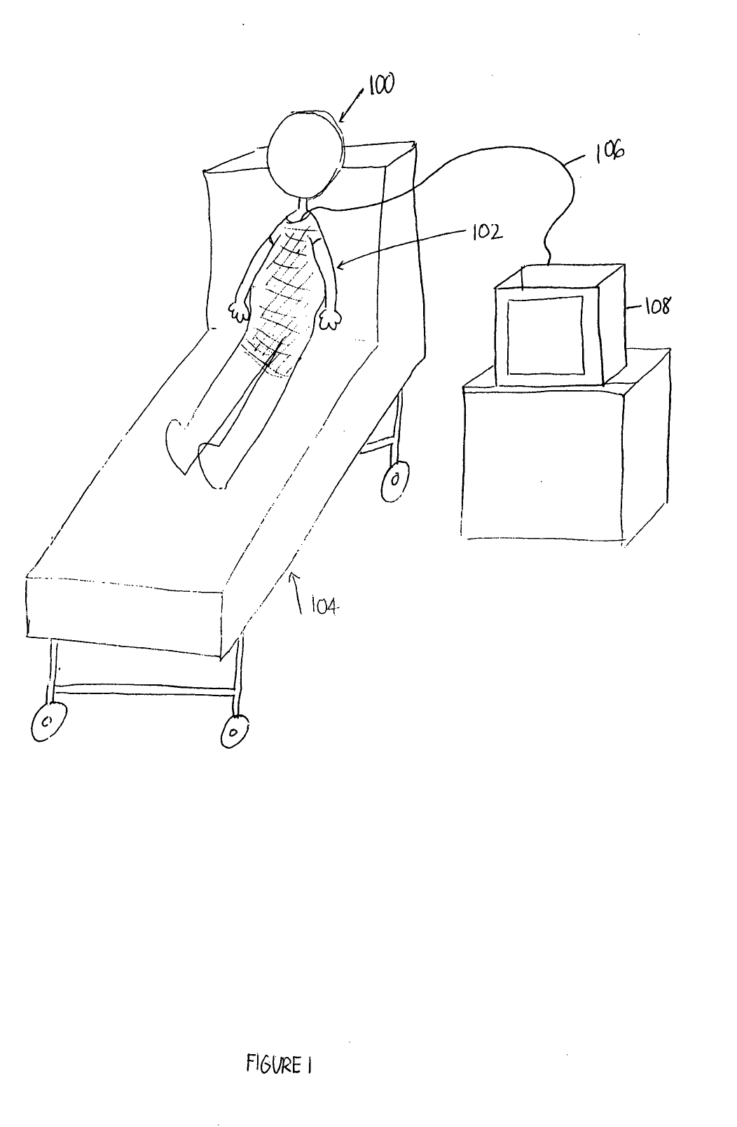

In the following description of the non-limiting embodiments, like reference numerals refer to like elements throughout. A first non-limiting embodiment will now be described with reference to FIG. 1.

As illustrated in FIG. 1, a patient 102 positioned on bed 104 and undergoing a seated neurosurgical procedure on area 100 may be connected to monitor 108 via connector 106. Monitor 108 may be configured to signal an alarm upon the occurrence of VAE.

While the patient 102 is illustrated undergoing a seated neurosurgical procedure in FIG. 1, other positions for neurosurgical procedures are within the scope of the invention. Similarly, other types of surgical procedures, such as (by way of non-limiting examples) hysterectomy, back surgery, abdominal procedures, liver transplantation, craniofacial surgery, head surgery, neck surgery, radical prostatectomy, retrograde pyelogram percutaneous nephrolithotomy, vascular catheterization, shunting, endoscopy, and transfusion of intravenous fluids under pressure without an air trap chamber are also within the scope of the present invention. Of course, any surgery in which VAE may occur is within the scope of the present invention.

While the following explanation focuses on the risks associated with central venous catheter aspiration, it should not be considered limiting of the present invention. In other words, the present invention is equally applicable to other situations in which VAE may arise.

By way of explanation, central venous catheter aspiration may be used to aspirate air in the central venous system and right atrium during seated surgical procedures. Aspiration, as used in the context of the present invention, refers to the process of removing fluids or gases from the body with a suction device.

Occurrence of an embolism may be recognized by audible air aspiration during catheter insertion or by fluoroscopic visualization of air within the right atrium or pulmonary artery. As noted above, such an embolism may be fatal to the patient 102. For example, a bolus of air could lead to an airlock on the right side of the heart, which could result in death.

As one aspect of the present invention, the Doppler catheter of the first non-limiting embodiment may be associated with the central venous catheter aspiration procedure. In the aspiration procedure, a catheter may be inserted into the vena cava via the jugular vein, for example. To detect VAE from surgical wounds in the venous system of the upper part of the body, detection may be made in the superior vena cava. For VAE resulting from surgical wounds in the lower part of the body, detection may preferably be performed in the inferior vena cava. However, other locations of detection known to those of skill in the art are also within the scope of the present invention.

To be useful for central venous catheter aspiration, it may be preferable that the Doppler catheter be of a size suitable to be positioned within a lumen of a catheter. The catheter may then be inserted in the vena cava via the internal jugular vein, for example. Once in the vena cava, the Doppler catheter may monitor the occurrence of VAE by monitoring the flow of blood during the aspiration procedure.

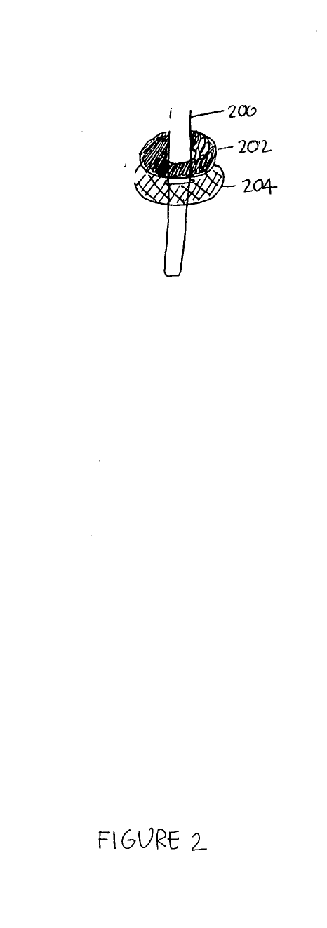

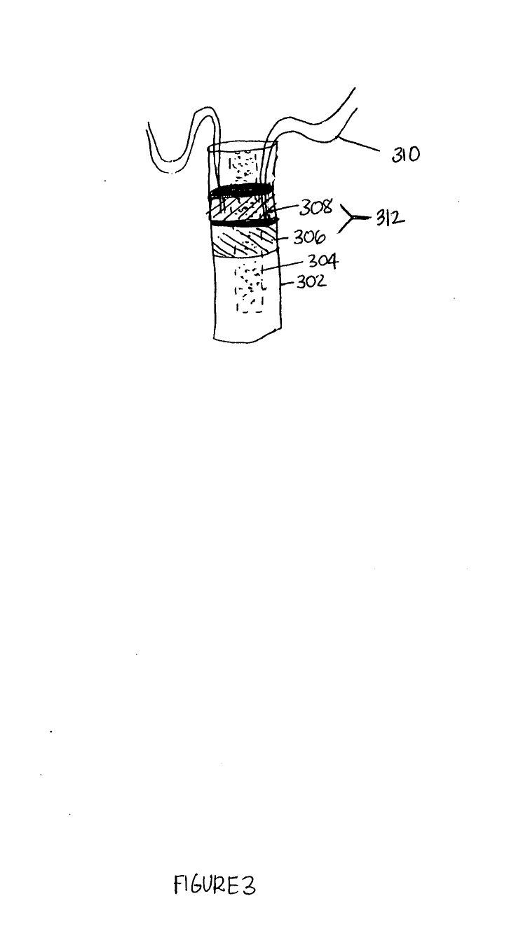

To this end, FIGS. 2 and 3 illustrate non-limiting exemplary embodiments of the Doppler catheter according to the present invention. As shown in FIG. 2, inner lumen 202 of a catheter may be at least partially surrounded by first piezoelectric element 202 and second piezoelectric element 204. Because first piezoelectric element 202 and second piezoelectric element 204 may be the same as or similar to the first and second piezoelectric elements 306 and 308 of FIG. 3, the first and second piezoelectric elements will be described in more detail below with reference to FIG. 3.

As illustrated in FIG. 3, leads 310 may be used to connect transmitter/receiver 312 to a flow monitor (illustrated, for example, in FIG. 1 as element 108). The transmitter/receiver 312 may be formed of a stacked piezoelectric material. The piezoelectric material may be in the form of a PZT ceramic disk or a piezoelectric film, for example.

As illustrated in FIG. 3, elements 306 and 308, which form the transmitter/receiver, may be a stacked, concentric piezoelectric material. Elements 306 and 308 may be made of the same, similar, or different materials, as desired. Because elements 306 and 308 may interchangeably function as the transmitter and receiver, they are collectively referred to as transmitter/receiver 312. However, in a preferred configuration, the transducer element closest to the tip of the catheter (distal) may function as the receiver, while the transducer element nearest the syringe (proximal) may function as the transmitter. Transmitter/receiver 312 may be configured to receive a reflected or refracted ultrasound signal, to convert the ultrasound signal to an electrical signal, and to transmit the electrical signal to a monitor, such as monitor 108.

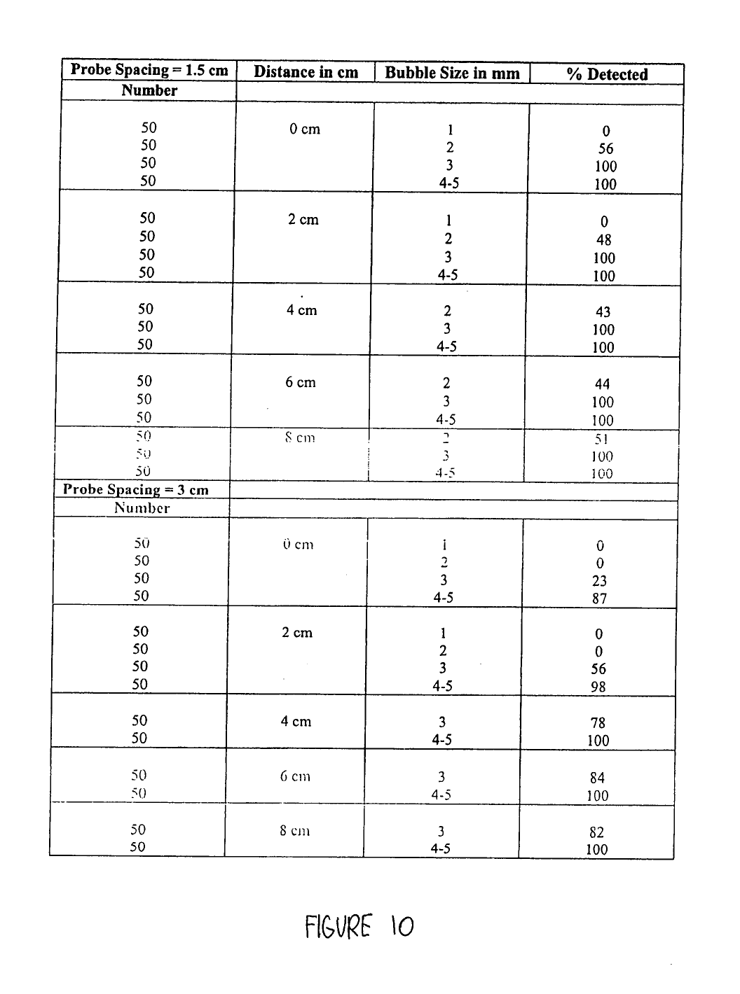

Although elements 306 and 308 are illustrated as positioned adjacent to each other in FIG. 3, other configurations are within the scope of the present invention. In fact, by changing the distance between the two transducers, it may be possible to maximize the sensitivity of the Doppler catheter of the present invention. By way of non-limiting example, when the distance between elements 306 and 308 is set to 1.5 cm, it is possible to detect bubbles (e.g., emboli) that have a diameter of 2.0 mm or greater. When the distance between elements 306 and 308 is set to 3 cm, it is possible to detect bubbles that are 3 mm or greater in diameter. Similarly, when the spacing between the transducers is 6 cm, it is possible to detect bubbles having a minimum diameter of 5 mm. These levels of sensitivity were determined at least in part, based on the experimental results shown in FIG. 10. As shown in FIG. 10, the spacing between transducer elements for the first set of probes was approximately 1.5 cm, and the spacing between transducer elements for the second set of probes was approximately 3 cm.

As further illustrated in FIG. 3, the transmitter/receiver 312 may be positioned inside double lumen catheter 302, and may surround at least a portion of aspiration lumen 304. While the catheter illustrated in FIG. 3 is a double lumen catheter, other catheters known to those of skill in the art are within the scope of the present invention.

To minimize the amount of redesign required for incorporating the present invention into existing systems, it may be preferable that the Doppler catheter is configured to be compatible with resonant frequencies from approximately 2.25 MHz to approximately 9 MHz. These frequencies are the most common frequencies in use today. However, should other frequency ranges become preferred or more prevalent, the Doppler catheter of the present invention could be configured to be compatible with these other frequency ranges. Because lower frequencies generally obtain higher penetration depth, it may be preferable to configure the Doppler catheter to operate close to the 2.25 MHz range.

A number of companies manufacture optical interfaces that may be incorporated into the present invention. For example, Hewlett Packard, Siemens, and General Electric (among others) all manufacture suitable interfaces. The optical interface may be used to convert the electrical signal to a digital light signal. When received at the acoustical box (flow detector box), the digital light signal is converted back to an electrical signal.

The electrical components may also preferably be isolated from the body of patient 102. Specifically, leads 310 may preferably be electrically isolated, especially when inside the body of patient 102. The electrical isolation of the leads may be achieved, for example, by coating the leads 310 with a non-conducting polymer. A non-limiting example of the non-conducting polymer is polyurethane. The coating may occur where leads 310 are connected to the catheter assembly. In this way, it is possible to isolate the patient from microshock due to current leakage.

To facilitate insertion to the internal jugular vein without restricting blood flow, it may be preferable that a maximum outer diameter of the catheter not exceed about 4 mm. It may also be preferable that the transducer element of the Doppler catheter be accommodated in a 7 Fr. catheter, which has an outer diameter of about 2.3 mm.

To maximize compatibility with existing systems, the transducer may be compatible with existing flow detector boxes, such as a flow detector box manufactured by Parks Medical Electronics (Aloha, Oreg.). Of course, other flow detector boxes are within the scope of the present invention.

If modifications to the signal output by the transducer are useful, it may be preferable that a secondary circuit be provided to interface with the flow detector box. This secondary circuit may be provided internal or external to the transducer. For example, the secondary circuit may be physically positioned between the transducer and the flow detector box. Alternatively, the secondary circuit may be added to the flow detector box, as well as to the transducer.

An added advantage of the Doppler catheter of the present invention is that the Doppler catheter may be configured to view a cross-section of the blood vessel at a location upstream of the aspiration site. By viewing an upstream location, it is possible to more rapidly detect and aspirate the VAE. As a result, it is possible to prevent the VAE from reaching the heart and causing death.

To enable visualization of the entire cross-sectional area of the blood vessel (which typically has an inner diameter of about 0.7 cm to about 1.1 cm), the Doppler catheter may penetrate to a desired depth. This depth may be, for example, at least about 6 mm for a probe having an outer diameter of about 4 mm. Of course, as the size of the probe varies, the corresponding depth may also vary.

As an alternative, it is possible to process a signal from the Doppler catheter to enable a visual display of the bubbles in the vein. This may be achieved by adapting the Doppler catheter to interface with devices currently used or similar to those currently used for echocardiography or obstetrical sonography. Examples of such devices include devices manufactured by SonoSite, Hewlett Packard, and Accuson (among others).

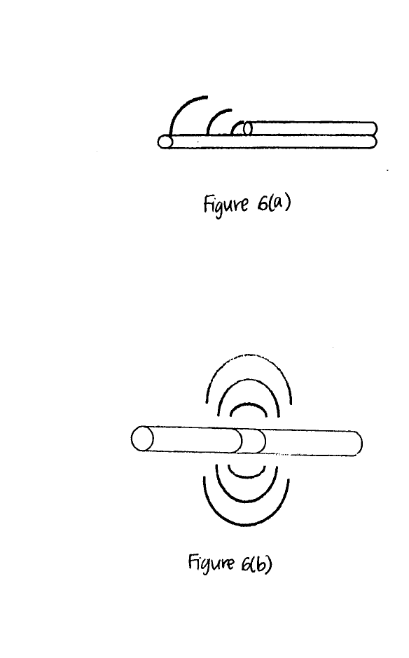

According to the present invention, several different configurations of the Doppler catheter are possible. The second and third non-limiting embodiments both have a field of view parallel to the vessel wall. The second non-limiting embodiment views upstream of the Doppler catheter. The third non-limiting embodiment views downstream of the Doppler catheter. A non-limiting example of the configurations of the second and third non-limiting embodiments may be seen in FIG. 6(a). To achieve the different upstream and downstream views, the configuration in FIG. 6(a) may be rotated approximately 180°. However, the views for both the second and third non-limiting embodiments may be at least partially blocked by the Doppler catheter.

Accordingly, so that the view is not hindered by the Doppler catheter, the fourth non-limiting embodiment may include a piezoelectric material having a 360° field of view. The field of view may be focused radially outwardly from the Doppler catheter. The radial configuration enables an improved field of view as compared with the second and third embodiments.

The fourth non-limiting embodiment includes several possible configurations, one of which is illustrated in FIG. 6(b). To obtain the 360° view, it is possible to use a ceramic piezoelectric element and/or a piezoelectric film. A non-limiting example of the material that may be used for the ceramic piezoelectric element is lead zirconate titanate. The piezoelectric film may include, for example, a 122 μm silver ink sheet. Another non-limiting example of the material that may be used for the film is polyvinylidene fluoride (PVDF). PVDF may be formed in thin, flexible sheets and may be manually formed into a cylinder.

To obtain a 360° field of view, it may be desirable to have a maximum diameter of about 0.7 cm. So that the Doppler catheter of the present invention may be able to detect emboli within the lumen of the jugular vein or vena cava, the Doppler catheter of the present invention may be able to detect emboli that range in size between approximately 0 cm and 1.5 cm. Preferably, the diameter of the Doppler catheter is approximately 4 mm.

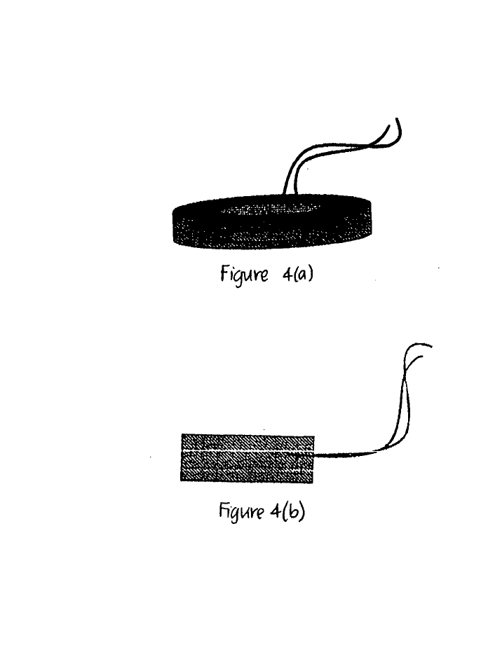

For a piezoelectric film, an example of which is illustrated in FIG. 4(b), the resonant frequency may be calculated using the following formula: fᵣ=v/2t, where v is the speed of sound through the surrounding medium and t is the thickness of the film. Based on this formula, an exemplary thickness of the PVDF film in the fourth embodiment could be approximately 300 μm for a resonating frequency of 2.25 MHz. For a resonating frequency of 9 MHz, the thickness could be approximately 85 μm.

By contrast, the resonant frequency of a ceramic piezoelectric element may depend on the material from which the ceramic is made. A non-limiting example of a ceramic piezoelectric element configuration is illustrated in FIG. 4(a).

Each material has a thickness constant (Nₜ). For a cylindrical tube, the resonating frequency may be calculated using the formula: fᵣ=2Nₜ/(Dₒ−Dᵢ), where Dₒ represents the outer diameter and Dᵢ represents the inner diameter of the cylinder. To satisfy the desired design parameters, the inner diameter may preferably be large enough to accommodate the aspirator. For example, the inner diameter may be approximately 2.3 mm. Therefore, for a resonant frequency of 2.25 MHz, the thickness frequency constant may be within the range of 1900 Hz-m.

Another factor in the configurations of all of the non-limiting embodiments is the height of the piezoelectric element. If the height is less than 1.5 times the diameter of the cylinder, the behavior of the piezoelectric elements may become unpredictable. Therefore, at a minimum, the height of the cylinder could be approximately 6 mm if the outer diameter is 4 mm.

All of the embodiments may be configured to generate a sufficient signal, rather than the “best” signal. In other words, while the internal Doppler catheter of the present invention may generate a signal that is significantly improved over the external configurations of the related art, a generated signal may nonetheless be sufficient if it is at least as strong as the signal generated by the external configuration of the related art.

Although certain non-limiting examples of piezoelectric materials have been provided herein for explanatory purposes, it should be noted that other materials are also within the scope of the present invention. The piezoelectric transducers may be secured to the catheter using materials such as polyurethane, polyamide, polyimide, Teflon, a material of which the catheter itself is made, or another material known to those of skill in the art.

A factor that may be considered when selecting the piezoelectric elements is the tendency of the material to heat when operated at the resonant frequency. Excessive heating of the transmitting element could potentially harm patient 102 as well as the Doppler catheter itself. As long as the piezoelectric element's temperature remains within a safe operating range, any suitable material may be selected therefor, such that the Doppler catheter temperature may preferably not exceed 105° F.

Another factor that may be considered when selecting the material for the piezoelectric element is the human body's likelihood of experiencing an allergic reaction or the likelihood of rejection. To minimize the risk of reaction by the body, the transducers may be coated with a non-reactive substance, such as polyurethane, polyamide, Teflon, or the like. Other non-reactive coatings known to those of skill in the art are also within the scope of this invention.

To minimize the cost of the Doppler catheter, it may be preferable that the Doppler catheter be disposable. To this end, the following materials may be particularly beneficial in terms of minimizing cost: polyurethane, Teflon, nylon, polyvinylchloride, and polyethylene. However, other materials known to those of skill in the art are also within the scope of the present invention.

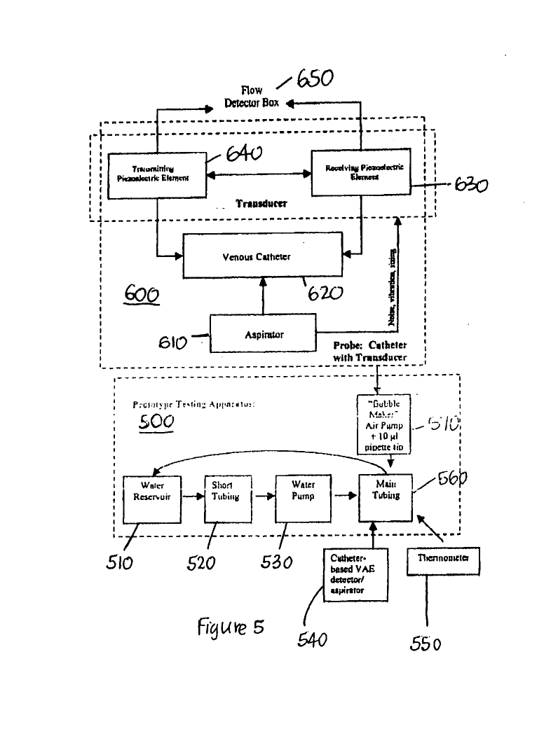

An exemplary testing system into which the Doppler catheter of the present invention may be incorporated is illustrated in FIG. 5. As shown in FIG. 5, the prototype testing apparatus 500 may include water reservoir 510, short tubing 520, water pump 530, catheter-based VAE detector/aspirator 540, thermometer 550, main tubing 560, and bubble maker air pump with 10 μm pipette tip 570.

As further illustrated in FIG. 5, the probe 600 may include aspirator 610, venous catheter 620, receiving piezoelectric element 630, transmitting piezoelectric element 640, and flow detector box 640. The features of probe 600 may be analogous to those set forth above with respect to FIG. 3.

By using the testing system of FIG. 5, it is possible to configure a Doppler catheter according to the present invention that satisfies desired parameters. In more detail, the prototype testing apparatus may be used to determine whether or not a particular configuration of the Doppler catheter of the present invention satisfies desired design parameters, such as a desired frequency range, sensitivity, or penetration depth, by way of non-limiting example.

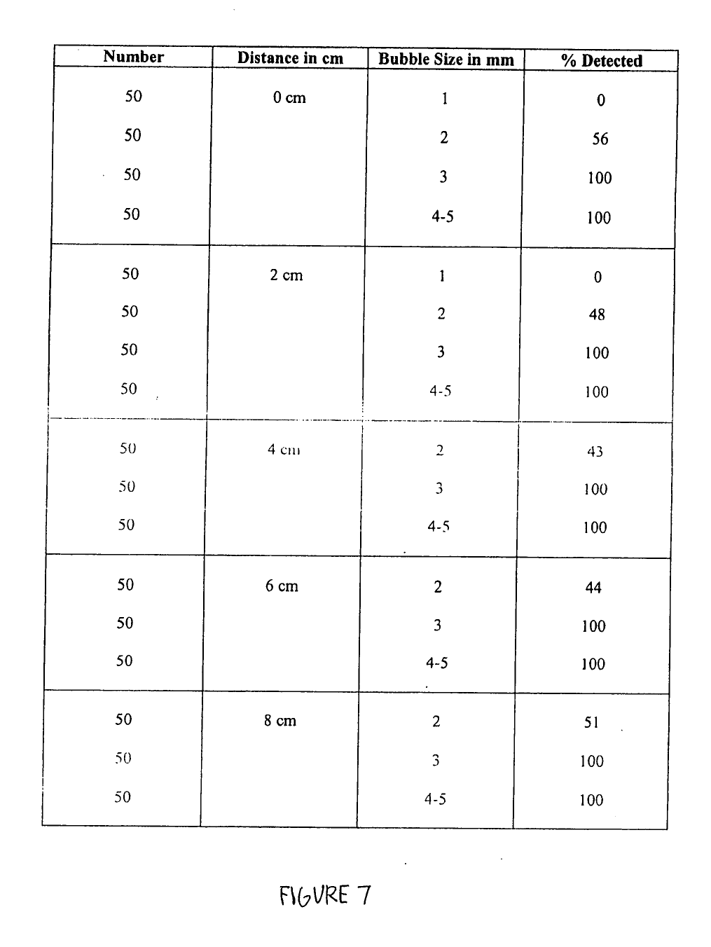

FIGS. 7-10 illustrate test results achieved using the exemplary testing system of FIG. 5. FIG. 7 provides an explanation of the relationship between the distance from the bubble source (e.g. the site of air entry) at which the catheter is positioned, the size of the bubbles, and the percentage of the bubbles detected. As can be seen from FIG. 7, sensitivity improves when the distance between the bubble source and the Doppler assembly increases.

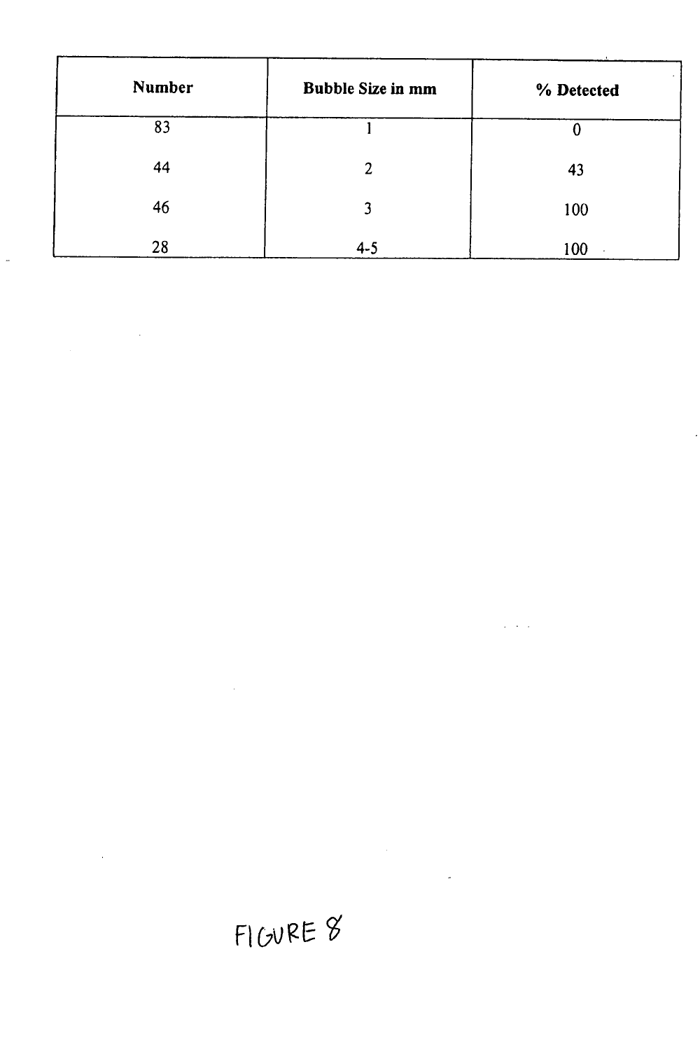

FIG. 8 provides an illustration of the effect of the bubble size on the percentage of the bubbles detected. According to the results shown in FIG. 8, detection rates increase as bubble size increases.

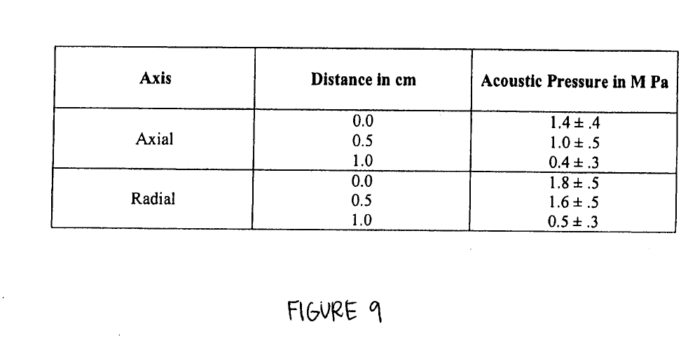

Measured amounts of acoustic pressure are shown in FIG. 9. The higher the acoustic pressure, the more likely the Doppler assembly is to cause cavitation. In other words, at higher acoustic pressures, the Doppler catheter may itself cause bubbles to occur. These pressures were measured according to the distances specified in FIG. 9, where the distances represent distances between the Doppler catheter and a hydrophone. The hydrophone (or analogous device) may be used to measure acoustic pressure.

FIG. 10 illustrates the relationship between piezoelectric element spacing, the distance from the bubble source, bubble size, and the percentage of bubbles detected. As shown in FIG. 10, the greatest detection rates were achieved when the bubble size is larger, irrespective of the distance between piezoelectric elements and irrespective of the distance between the Doppler catheter and the bubble source.

Because blood clots tend to develop along the surface of a catheter when the catheter remains in the patient 102 for an extended period of time, it may be desirable to prepare the surface with an anti-coagulant agent, such as heparin. The catheter may be soaked in a solution containing the anti-coagulant, impregnated with the anti-coagulant, or prepared in another manner known to those of skill in the art.

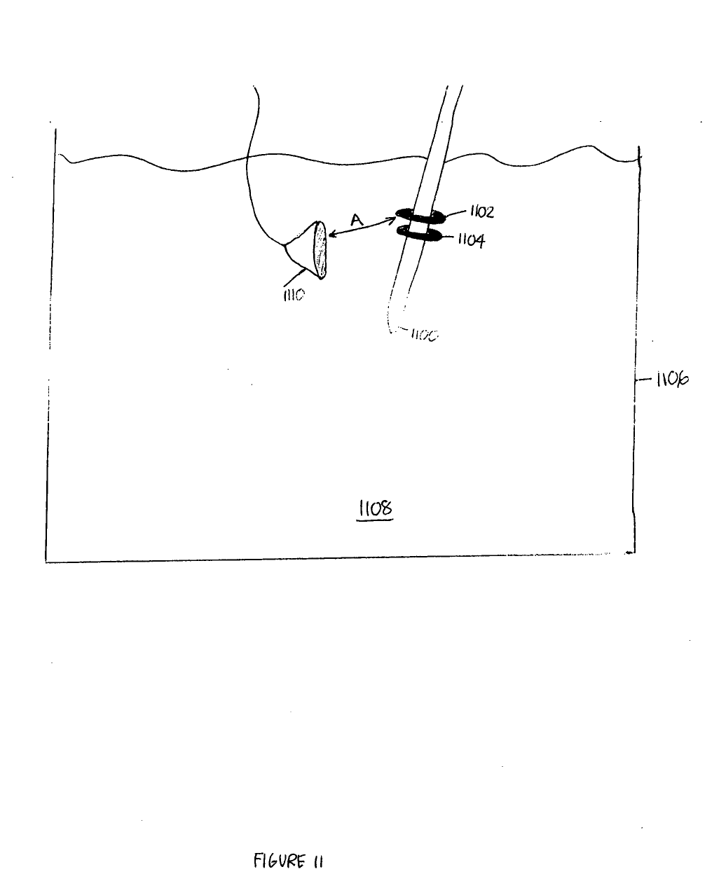

FIG. 9 illustrates certain aspects of the invention related to acoustic pressure. Acoustic pressure is a parameter that may be measured to determine the safety of the device. When the transducer operates above a certain pressure, it may cause the blood around it to boil or may cause cavitations or bubbles. Cavitation generally is more likely to occur at pressures greater than 2 MPa. The pressures illustrated in FIG. 9 are below a level likely to cause cavitations. The distance, measured in cm, is a distance between the transducer to the hydrophone. This distance is illustrated by A, for example, in FIG. 11.

The hydrophone is a microphone that may be used to detect acoustic pressure in a liquid. Of course, other means of detecting acoustic pressure known to those of skill in the art are also within the scope of the present invention.

As shown in FIG. 11, a hydrophone 1110 may be positioned at a distance A from a Doppler catheter 1100. Doppler catheter 1100 includes piezoelectric elements 1102 and 1104, which may be selected from any of the piezoelectric elements described above. In the exemplary system of FIG. 11, the Doppler catheter 1100 and the hydrophone 111O are positioned in a tank 1106 of water 1108.

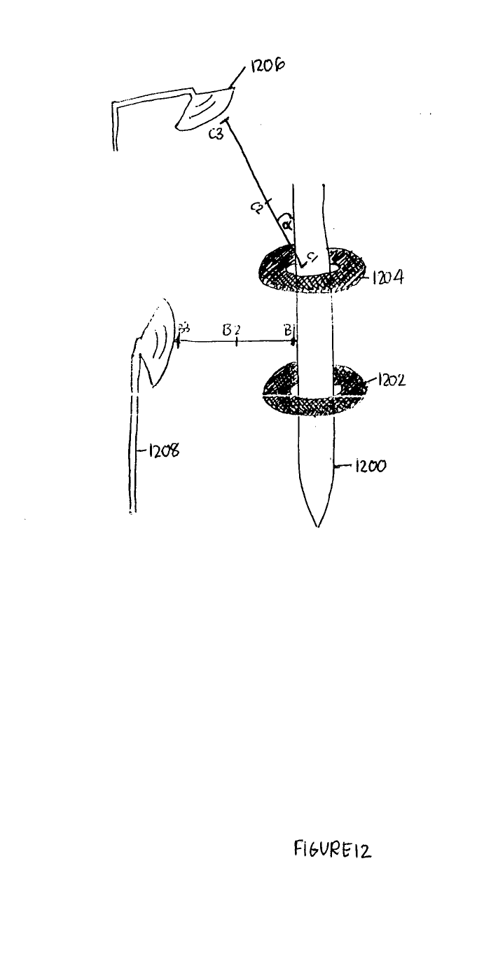

FIG. 12 provides a more detailed explanation of an exemplary interrelationship between hydrophones 1206 and 1208 and the Doppler catheter 1200. As shown in FIG. 12, Doppler catheter 1200 includes piezoelectric elements 1202 and 1204, which may be selected from any of the piezoelectric elements described above. In the configuration of FIG. 12, hydrophone 1206 measures the pressure at points C1, C2, and C3. These points may be 0.0 cm, 0.5 cm, and 1.0 cm from the Doppler catheter 1200, for example. In the configuration of FIG. 12, hydrophone 1206 is positioned to measure the pressure at an angle α. In this non-limiting example, angle α may have a value of approximately 15° relative to the axial axis (the axis that is perpendicular to the flat surface of the Doppler assembly). Hydrophone 1208 measures pressures at points B1, B2, and B3. These positions may be set, for example, to be at distances of 0.0 cm, 0.5 cm, and 1.0 cm from the Doppler catheter 1200.

As an example, the hydrophone may include a transducer. The transducer may include, for example, a piezoelectric film. Generally, a hydrophone is calibrated using known pressures, and the output of the hydrophone (an electrical current) may be converted into a value corresponding to a measured pressure.

Although certain embodiments have been illustrated herein, the features of these embodiments should not be considered limiting of the present invention. Of course, various modifications to the present invention, and equivalents thereof are included within the scope of the appended claims and their equivalents.

What is claimed is:

1. A Doppler catheter assembly, comprising:
a catheter having an inner lumen and an outer lumen;
a first piezoelectric element; and
a second piezoelectric element,
wherein the first piezoelectric element is adapted to be positioned proximate to the second piezoelectric element about the inner lumen, thereby forming a transmitter/receiver.

2. The Doppler catheter assembly according to claim 1, wherein at least one of the first and second piezoelectric elements is formed of at least one of a PZT ceramic disk and a piezoelectric film.

3. The Doppler catheter assembly according to claim 1, wherein the inner lumen includes an aspiration lumen.

4. The Doppler catheter assembly according to claim 1, wherein the Doppler catheter assembly is configured to operate at frequencies between approximately 2.25 MHz and approximately 9 MHz.

5. The Doppler catheter assembly according to claim 1, wherein the Doppler catheter assembly is at least partially coated with an electrically isolating material.

6. The Doppler catheter assembly according to claim 5, wherein the electrically isolating material includes a non-conducting polymer.

7. The Doppler catheter assembly according to claim 6, wherein the electrically isolating material includes a polyurethane.

8. The Doppler catheter assembly according to claim 1, wherein a maximum outer diameter of the outer lumen is 4 mm.

9. The Doppler catheter assembly according to claim 1, wherein the Doppler catheter assembly is configured to detect emboli having diameters between approximately 0 cm and approximately 1.5 cm.

10. The Doppler catheter assembly according to claim 1, wherein at least a portion of the Doppler catheter assembly includes an anti-coagulant.

11. A Doppler catheter assembly, comprising:
a catheter having an inner lumen and an outer lumen;
a first means for sensing; and
a second means for sensing,
wherein the first means for sensing is adapted to be positioned proximate to the second means for sensing about the inner lumen, thereby forming a transmitter/receiver.

12. The Doppler catheter assembly according to claim 11, wherein at least one of the first and second means for sensing includes a piezoelectric element.

13. The Doppler catheter assembly according to claim 12, wherein the piezoelectric element is formed of at least one of a PZT ceramic disk and a piezoelectric film.

14. The Doppler catheter assembly according to claim 11, wherein the inner lumen includes an aspiration lumen.

15. The Doppler catheter assembly according to claim 11, wherein the Doppler catheter assembly is configured to operate at frequencies between approximately 2.25 MHz and approximately 9 MHz.

16. The Doppler catheter assembly according to claim 11, wherein the Doppler catheter assembly is at least partially coated with an electrically isolating material.

17. The Doppler catheter assembly according to claim 16, wherein the electrically isolating material includes a non-conducting polymer.

18. The Doppler catheter assembly according to claim 17, wherein the electrically isolating material includes a polyurethane.

19. The Doppler catheter assembly according to claim 11, wherein a maximum outer diameter of the outer lumen is 4 mm.

20. The Doppler catheter assembly according to claim 11, wherein the Doppler catheter assembly is configured to detect emboli having diameters between approximately 0 cm and approximately 1.5 cm.

21. The Doppler catheter assembly according to claim 11, wherein at least a portion of the Doppler catheter assembly includes an anti-coagulant.
